(19) United States
(12) Patent Application Publication
(10) Pub. No.: US 2025/0049359 A1
(43) Pub. Date: Feb. 13, 2025

(54) POINT-OF-CARE MULTIPLEXING BIOSENSOR ARRAY AND SCALABLE METHOD OF MANUFACTURE

(71) Applicant: CARNEGIE MELLON UNIVERSITY, Pittsburgh, PA (US)

(72) Inventors: Rahul Panat, Pittsburgh, PA (US); Burak Ozdoganlar, Pittsburgh, PA (US); Toygun Cetinkaya, Pittsburgh, PA (US); Chunshan Hu, Pittsburgh, PA (US)

(73) Assignee: CARNEGIE MELLON UNIVERSITY, Pittsburgh, PA (US)

(21) Appl. No.: 18/931,335

(22) Filed: Oct. 30, 2024

Related U.S. Application Data
(63) Continuation-in-part of application No. 18/848,255, filed on Jan. 1, 0001.
(63) Continuation-in-part of application No. 18/013,951, filed on Dec. 30, 2022.
(60) Provisional application No. 63/546,310, filed on Oct. 30, 2023.
(60) Provisional application No. 63/321,425, filed on Mar. 18, 2022.
(60) Provisional application No. 63/047,368, filed on Jul. 2, 2020.
(60) Provisional application No. 63/050,182, filed on Jul. 10, 2020.
(60) Provisional application No. 63/076,174, filed on Sep. 9, 2020.

(51) Int. Cl. A61B 5/1468; A61B 5/1486

(52) U.S. Cl. CPC A61B 5/1468; A61B 5/1486

(57) ABSTRACT

A multiplexing electrochemical point-of-care biosensing device that provides picomolar level accuracy and high selectivity and which requires only seconds to provide the response. The biosensing device is capable of being mass manufactured due to a molding process used to fabricate the working electrode of the device, which consists of an array of micropillars coated with a biosensitive material.

### RELATED APPLICATIONS

This application claims the benefit of U.S. Provisional Patent Application No. 63/546,310, field Oct. 30, 2023.

This application is also a continuation-in-part filing of U.S. patent application Ser. No. 18/848,255, filed Sep. 18, 2024, which is a filing under 35 U.S.C. § 371 of PCT Application PCT/US2023/15519, filed Mar. 17, 2023, which claims the benefit of U.S. Provisional Patent Application No. 63/321,425, filed Mar. 18, 2022.

This application is also a continuation-in-part filing of U.S. patent application Ser. No., 18/013,951, filed Dec. 30, 2022, which is a filing under 35 U.S.C. § 371 of PCT Application PCT/US2021/040302, filed Jul. 2, 2021, which claims the benefit of U.S. Provisional Patent Applications Nos. 63/047,368, filed Jul. 2, 2020, 63/050,182, filed Jul. 10, 2020 and 63/076,174, filed Sep. 9, 2020.

The contents of these priority applications are incorporated herein their entireties.

### FIELD OF THE INVENTION

This invention is related to the detection of biomolecules and, in particular, is directed to devices for use in the field at the point-of-care and which can provide accurate and timely results.

### BACKGROUND

Rapid, accurate, and selective detection of biomolecules is critical for a range of medical applications, including the detection of (1) pathogens and the associated antibodies; (2) biomarkers that indicate the biological status of the healthy individuals for, e.g., sports medicine; (3) biomarkers that indicate disease conditions, e.g., cancer or sexually-transmitted infections and diseases; (4) specific nucleic acids (RNAs and DNAs), and (5) harmful bioagents (e.g., bioweapon components), to name a few.

Generally, high accuracy and selectivity are only accomplished using laboratory test procedures. For instance, qPCR is a gold standard for detecting antibodies and antigens, capable of detecting picomolar or better concentrations. Such high accuracy and selective sensing are critical for the detection of infectious diseases to prevent catastrophic pandemics. However, laboratory testing takes a long time and requires the samples to be transferred, in an intact condition, from the collection point to a laboratory. Moreover, special equipment and training are required to conduct the testing procedures. Therefore, although laboratory tests can provide outstanding detection accuracy and selectivity, obtaining the response may take many hours to days. In many cases, this delay critically affects the effectiveness of the testing and, therefore, effective control of diseases, contagions, etc.

An alternative to laboratory testing is to use point-of-care (POC) devices. Such devices eliminate the need for the samples to be transferred to a laboratory and can provide the response within tens of minutes. There are many types of POC devices, from paper-based systems (e.g., COVID antigen tests) to those that use an electrochemical sensing approach. However, the accuracy (sensitivity) of the state-of-the-art POC devices is orders of magnitude below the laboratory tests, and their rate of false negatives and positives is considerably high. Furthermore, most current POC devices still require 15-30 mins to respond (which is still a relatively long time for many cases) and can only provide binary (yes or no) responses rather than quantitative results.

The prior art describes a POC device that uses a 3D array of micropillars as the basis of the sensing element. Such a device is described in U.S. Pub. Pat. App. 2023/0287226, the contents of which are incorporated herein by reference. In the described prior art device, the 3D array of micropillars must be created using a 3D printing process, using, for example, an Aerosol Jet machine. There are two disadvantages to this process, First, the process is capable of printing only a limited number of arrays in a given time period, making mass production of the device difficult. Second, the 3D printing process requires the use of nanoparticles of metal to create the array, which are expensive. Therefore, it would be desirable to be able to improve the production process to allow economical mass production of the device.

### SUMMARY OF THE INVENTION

Disclosed herein is a multiplexing and scalable electrochemical POC biosensing device that provides picomolar level accuracy and selectivity commensurate with laboratory testing and which requires only seconds to provide the response. Furthermore, in addition to binary response, the device can provide quantitative data to indicate the levels of biomolecules.

Further, the disadvantages of the prior art production method are improved upon in this invention through novel design modifications and an improved fabrication process, such as to provide for economically feasible large-scalable manufacturability of the device.

Specifically, a systems approach is used to redesign the biosensors, including scalable, micromolded electrodes (i.e., micropillar arrays), PCB designs to reproducibly and economically provide the required electronic infrastructure to support the components, and a molded microfluidic portion to enable the flow of the sample fluids. This approach enables the multiplexing of the sensor to be able to detect several different biomolecules simultaneously. Overall, the described invention will enable hundreds of thousands of sensors to be manufactured in a day, reducing cost and improving the accessibility of detecting the necessary biomarkers.

### BRIEF DESCRIPTION OF THE DRAWINGS

FIG. 1 is a schematic illustration of the 3D micropillar array forming the working electrode (WE) of an electrochemical cell for the detection of disease and other biomarkers. Also shown are the reference electrode (RE) and the counter electrode (CE).

FIG. 2 is a schematic illustration of the steps of the manufacturing process.

FIG. 3A is a SEM image of a single mastermold of the micropillar array. FIG. 3B is a SEM image of a mastermold having multiple micropillar arrays.

FIG. 4A is a SEM image of a single negative production mold of the micropillar array. FIG. 4B is a SEM image of a negative production mold having multiple micropillar arrays.

FIG. 5A is a SEM image of a green micropillar array prior to sintering. FIG. 5B is a SEM image of a micropillar array after sintering.

FIG. 6 is a SEM image of a single pillar, showing the porosity.

FIG. 7 is a SEM image of a gold-sputtered array

FIG. 8A is a schematic illustration of an electrochemical cell using printed circuit board (PCB) technology. FIG. 8B is a zoomed-in view of the microfluidic channel portion of the cell.

FIG. 9A is a schematic illustration of one embodiment of a microfluidic element used to deliver sample liquid to the testing chamber. FIG. 9B shows the microfluidic element of FIG. 9A in situ on a PCB.

FIG. 10 is a schematic illustration of the surface functionalization of the micropillars with pathogen (e.g., viral) proteins or other biomolecules.

FIG. 11 shows SEM images of the micropillars having a coating of a biosensitive material that can detect antibodies.

### DETAILED DESCRIPTION

In one embodiment of the invention, a POC device consists of an electrochemical biosensor of the type described in U.S. Pub. Pat. App. 2023/0287226 which has been productized by a scaled-up manufacturing process and mounted on a printed circuit board (PCB) for rapid deployment.

The device utilizes a micromolded working electrode in the form of an array of micropillars manufactured in accordance with a process described Int'l Pub. Pat. App. No. WO 2023/177878, entitled “Scalable Fabrication of Microdevices From Metals, Ceramics and Polymers”, which is incorporated herein by reference. Utilizing this manufacturing process for the working electrode allows for scaled-up production of the device, making it commercially viable as a product.

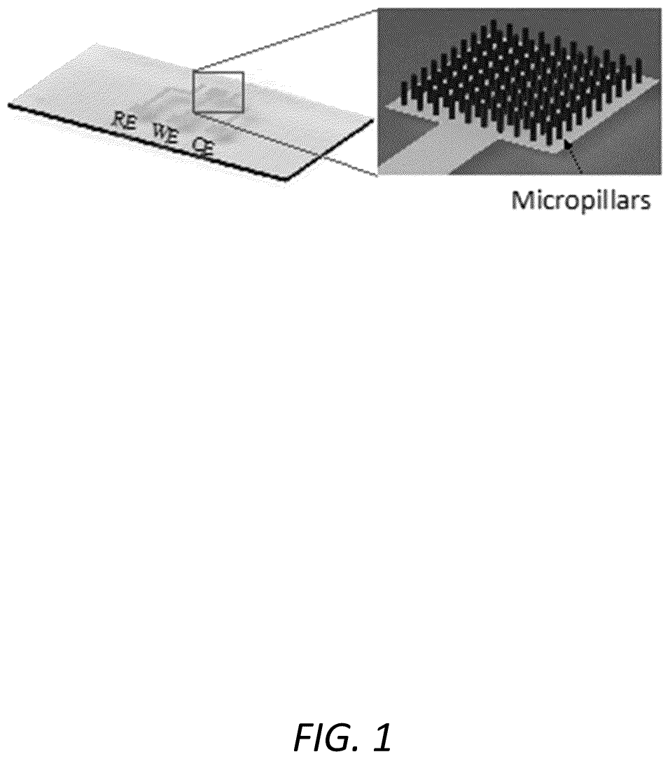

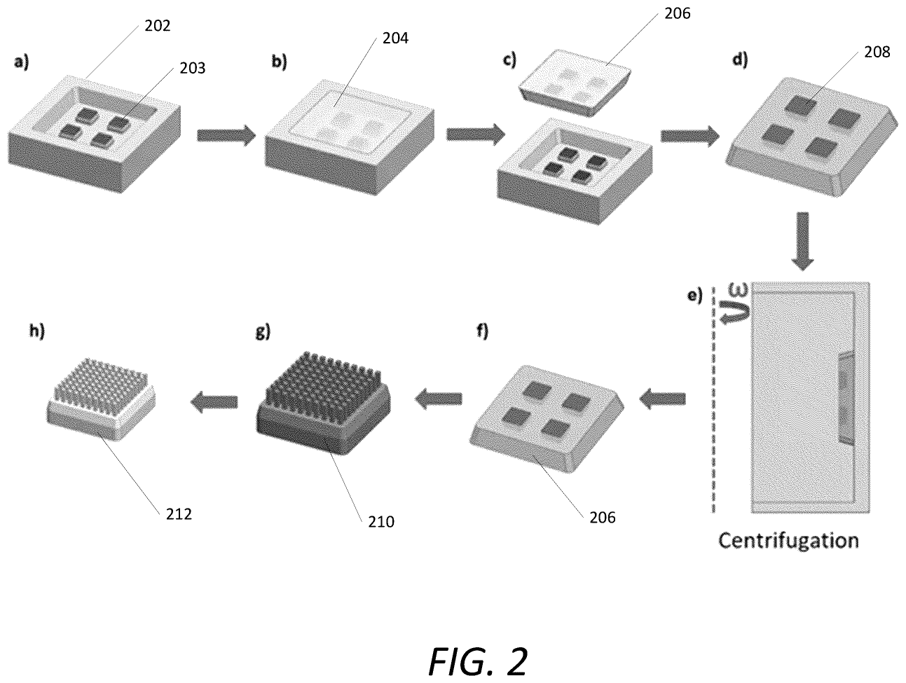

The working electrode consist of an array of micropillars, as shown in FIG. 1, that have been manufactured by a micromolding process, the steps of which are shown schematically in FIG. 2.

In step (a) of the process, a mastermold 202 is created, preferably using a mechanical micro milling process. In alternative embodiments, photolithography, electro-discharge machining, electro-chemical machining, or micro-3D printing may also be used to create mastermold 202. The body of mastermold 202 may be composed of a metal or a hard plastic, such as polymethyl methacrylate (PMMA). The surface of mastermold 202 is milled to create a wall for containment of the material from which the production mods is composed and to define arrays 203 and the micropillars in the arrays.

In some embodiments of the invention, the micropillars can have circular or rectangular cross section or a cross section of any other regular or irregular shape, depending on the application. Further, the number and arrangement of the micropillars within the array can be modified arbitrarily to ensure better sample flow and higher detection sensitivity. Similarly, the height of the pillars within an array can be modified. Additional flow distribution of flow focusing elements can also be integrated into the arrays to further optimize the sample flow.

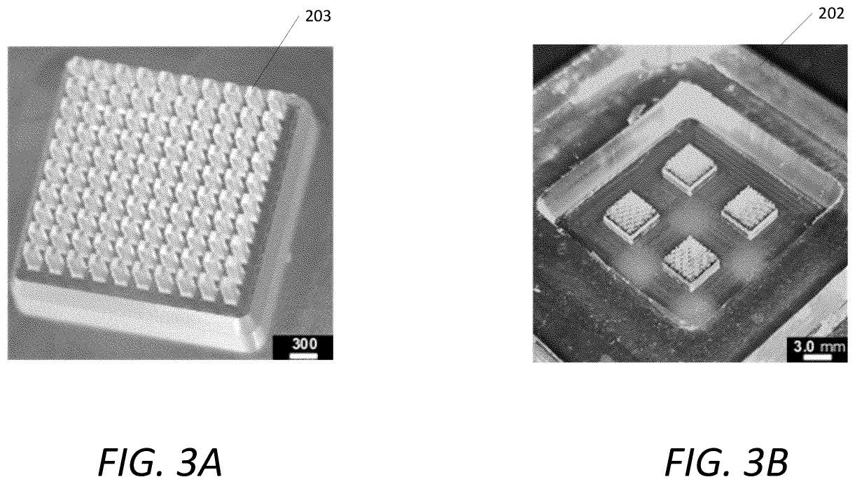

In a preferred embodiment, the micropillars of the arrays 203 on mastermold 202 have a square cross-sectional shape and are arranged in a regular 10×10 array. The micropillars of the mastermold, in the preferred embodiment, are approximately 150 μm on a side and 600 μm in height. FIG. 3A is a SEM image showing a close-up view of one array 203 of mastermold 202, while FIG. 3B shows mastermold 202 having 4 arrays 203 arranged thereon. Although FIG. 3B shows a mastermold having only four arrays, it should be realized that mastermolds having dozens or even hundreds of arrays defined thereon are desirable for mass production.

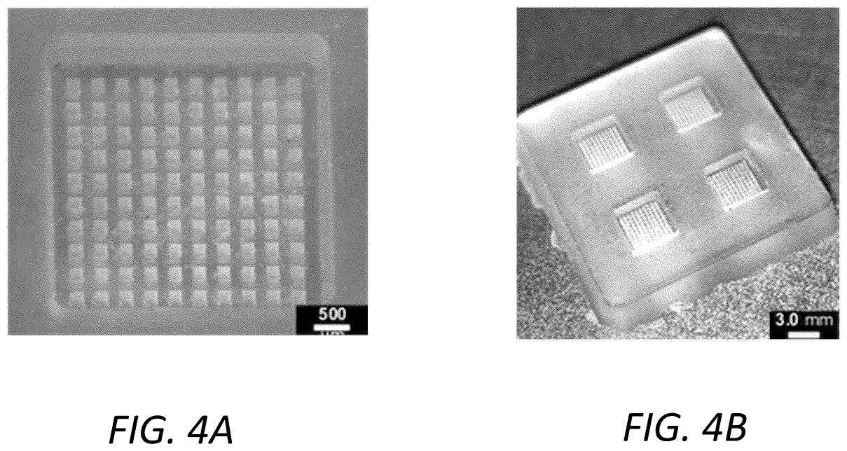

Steps (b) and (c) of the schematic of FIG. 2 show the preparation of the negative production mold 206 from the mastermold. In step (b), mastermold 202 is filled with the material 204 in liquid form of which the negative production mold 206 is to be composed. The material 204 may be an elastomer, for example, polydimethylsiloxane (PDMS) in preferred embodiments. The negative production mold 206 can be solidified by a curing process, which, in the case of PDMS, comprises hearting, for example, to 100° C. for 30 mins, or 125° C. for 20 mins. In step (c) of the process, the negative mold 206 is removed from mastermold 202. The removal process, in one embodiment, may be facilitated by a silanization process (or using other demolding agents) to allow easy separation of the negative, production molds 206 from mastermold 202. The process is similar to coating a lubricant layer on the mastermold. Production molds formed by this process may be reused many times. FIG. 4A is a SEM image showing a close-up view of one array 203 of an exemplary production mold 206, while FIG. 4B shows production mold 206 having 4 arrays 203 arranged thereon.

Steps (d-h) of FIG. 2 show the use of the production mold 206 to create the arrays 203 of micropillars. In step (d), a slurry 208 is deposited into the negative cavities of the mold 206. Each cavity of the production mold 206 is a negative shape of a single array 203.

The slurry consists of metal powder mixed with a binding agent. In various embodiment, the metal powder may comprise metal particles of any sinterable, conductive metal, for example, nickel, stainless steel, titanium, copper, copper alloys, gold, gold alloys, silver alloys etc. The size of the metal particles is generally dependent on the size of the micropillars being produced. For example, for a square micropillar having 125 μm sides, the metal particles may be in the 5 μm range. However, because it is desired that the micropillars have some porosity, the size of the metal particles should not be too small such as to reduce the porosity of the micropillars. The binding agent may be, for example, polyvinyl alcohol (PVA) mixed with a solvent, for example, water, although other binding agents, for example, glycerin or wax, may be used. The slurry 208 may comprise approximately 80% by weight of the metal particles and 20% by weight of the binding agent. When PVA is used as the binding agent, the binding agent may be approximately 30% by volume of PVA and 70% by volume of the solvent. Again, a silanization process may be used to facilitate removal of the arrays 203 from production mold 206.

Once the slurry 208 is placed in the cavities of production mold 206, production mold 206 is placed in a centrifuge, as shown in step (e) of FIG. 2. The centrifuge process serves to force slurry 208 into each pillar of each array 203. The production mold should be spun in the centrifuge for 5-15 minutes, depending on the consistency of slurry 208.

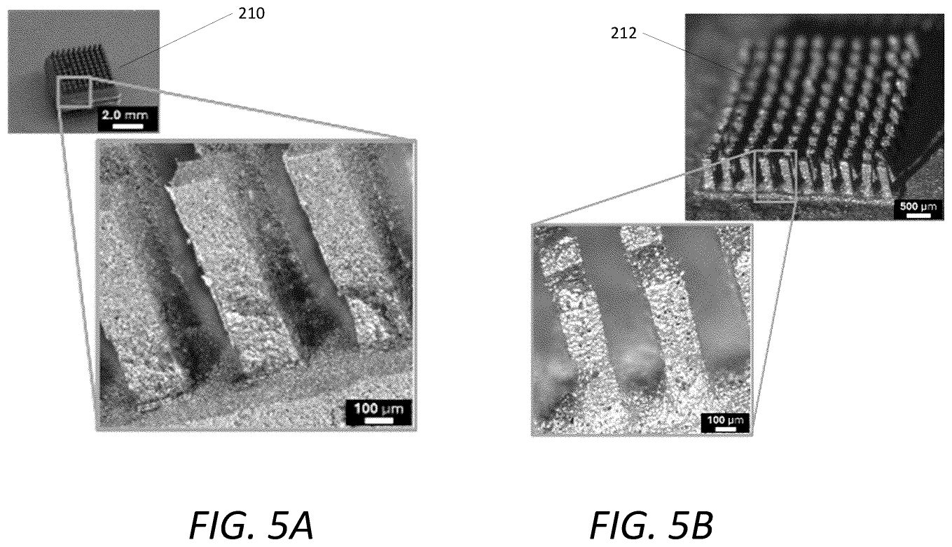

At step (f) individual arrays 203 are removed from production mold 206. The arrays may simply drop out of mold 206, or mold 206 may be placed into a centrifuge in a reversed orientation to facilitate the removal of the green arrays. At this point the arrays are referred to as “green” arrays 210, as shown in step (g) of FIG. 2. A SEM image of a green array is shown in FIG. 5A. The green arrays may then be subjected to a drying process by exposing the green arrays to a predetermined temperature for a predetermined period of time effective to evaporate the solvent, which may also be effective in facilitating the removal of the green arrays from the mold.

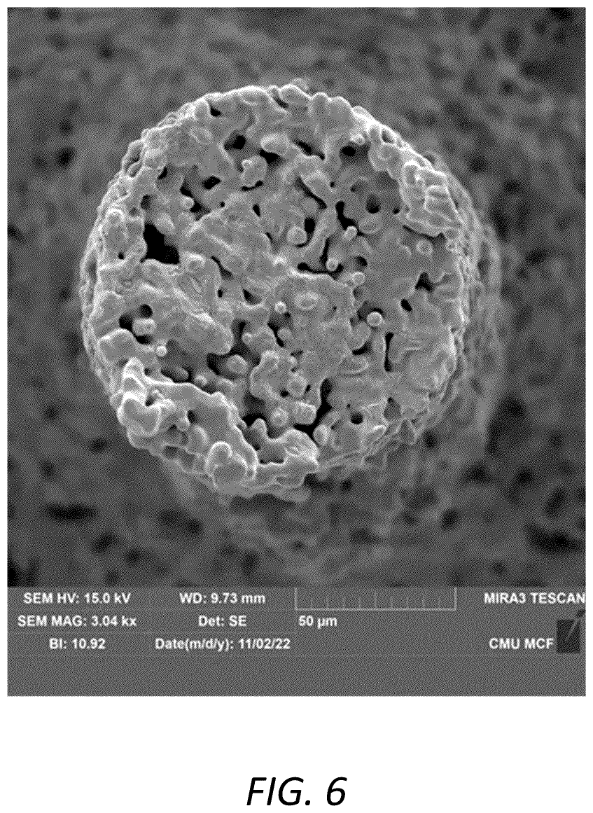

Thereafter, arrays 203 are sintered in step (h) of the process by exposure to a temperature range of 450° C.-600° C. at an average rate of 5° C./min (range: 1-10° C./min), atmospheric pressure, in the presence of argon gas, for a time period of about 30-120 mins. During the sintering process the binder molecules are burnt and removed from the arrays, thereby creating the porous voids in the micropillars, which at this point consist only of the metal, as shown in step (h) of FIG. 2. A SEM image of a sintered array 212 is shown in FIG. 5B. It should be noted that the micropillars have a tendency to shrink during the sintering process. For example, a 150 μm per side square micropillar may shrink to 125 μm. A SEM image of showing a close-up view of an individual micropillar is shown in FIG. 6, showing the porosity left behind when the binder is burnt off.

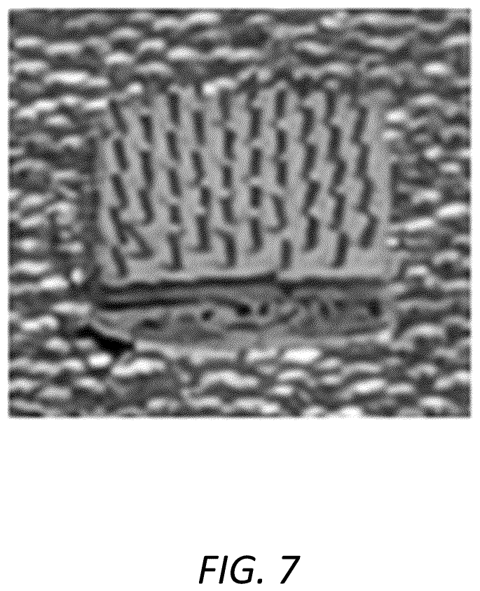

In some embodiments of the invention, the micropillars are coated with a secondary conductive coating (e.g., gold) to improve electrical conductivity, biomolecule attachment, or attachment of functional layers such as reduced graphene oxide (rGO). As an example, FIG. 7 shows a Ni micromolded working electrode coated with a thin (˜5-15 nm in thickness) layer of gold using a sputtering technique. In various embodiments, the conductive coating material can be applied by sputtering, chemical vapor deposition, physical vapor deposition, or electroplating. In various embodiments of the invention, the conductive coating material can be a metal, a conducting polymer or carbon, or any 1D or 2D material (e.g., carbon nanotubes, graphene, graphene oxide, MXenes) that can conduct electrons.

As may be realized by one of skill in the art, the micropillars may be designed to be of different shapes, sizes and heights, even within a single array. Further the density, arrangement and composition of the micropillars may vary from array to array. The porosity of the pillars may also vary from array to array and maybe effected by the ratio of the metal powder to the binder in the slurry or by the size of the metal particles in the metal powder.

After manufacture, the array of micropillars is used as the sensing element in the POC electromechanical biosensor. In one embodiment, the biosensor comprises an electronics module, a sensing module and a microfluidic module.

The electronics module comprises all components necessary to enable the operation of the biosensor and may include, for example, one or more of a power source (e.g., a battery), circuitry (i.e., one or more electronic components and/or a chip) for operating et biosensor and for detecting and comparing voltages on the working electrode, the counter electrode and the reference electrode, indicators controlled by the circuitry for outputting results of the analysis of the subject liquid (e.g., LEDs, digital read-outs, displays etc.), and means for offboarding the results of the analysis (e.g., Bluetooth® connectivity, electrical adaptors to external devices, etc.). All components of the biosensor are preferably mounted on a printed circuit board. The sensing module includes one or more working electrodes comprising arrays of micropillars, as described herein. The microfluidic module includes one or more microfluidic elements that serve to route a subject liquid(s) to one or more testing chambers, each testing chamber containing a working electrode (i.e., an array of micropillars), a reference electrode and a counter electrode.

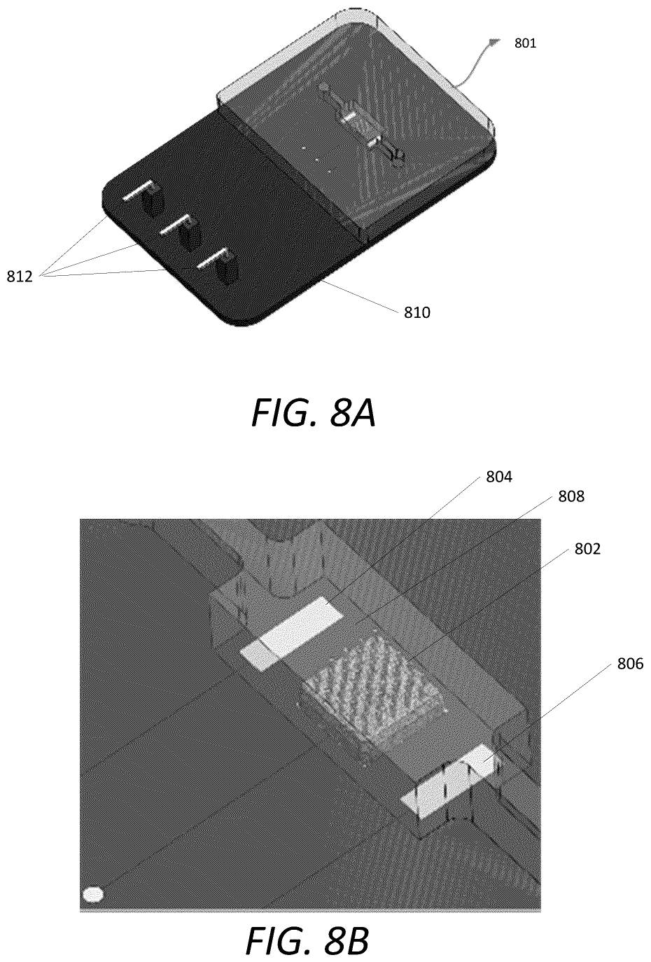

In one embodiment of the invention, the electrochemical biosensor is mounted on a printed circuit board 810, as shown in FIGS. 8(A-B), showing working electrode 802, counter electrode 804 and reference electrode 806, made using the PCB technology. PCBs 810, designed specifically for the POC biosensor, function as a low-cost, reproducible, and multifunctional device for sensing one or multiple biomolecules. The same PCB-based device can be designed to incorporate multiple electrode arrays and additional provisions for offboarding the sensor output (e.g., LED lights, digital read-outs, Bluetooth® connectivity, electrical connectors to external devices, etc.). In the exemplary embodiment shown in FIG. 8A, electrical connectors 812 are the means for offloading the sensor output.

In some embodiments of the invention, the device includes a microfluidic element 808 which is designed specifically to allow optimal flow of the sample liquid over the working electrode 802, the counter electrode 804, and the reference electrode 806. FIGS. 8(A-B) show such a microfluidic element 801 applied over the electrochemical cell and used to introduce the liquid to testing chamber 808 be tested. In some embodiments of the invention, the microfluidic element 801, is composed of an elastomeric material such as PDMS using a molding technique. In some embodiments, a hole is created on the microfluidic element 801 to introduce the sample fluid to the device. The microfluidic element 801 may include provisions to propel the liquid by, for example pressure (such as a thumb push portion), vacuum (a chamber opened at will), or an absorbent portion with a sacrificial liquid absorbed by the absorbent portion to create a vacuum. Auxiliary fluids and chemicals (e.g., PBS) for dilution, activation, etc., may be incorporated into microfluidic element 801.

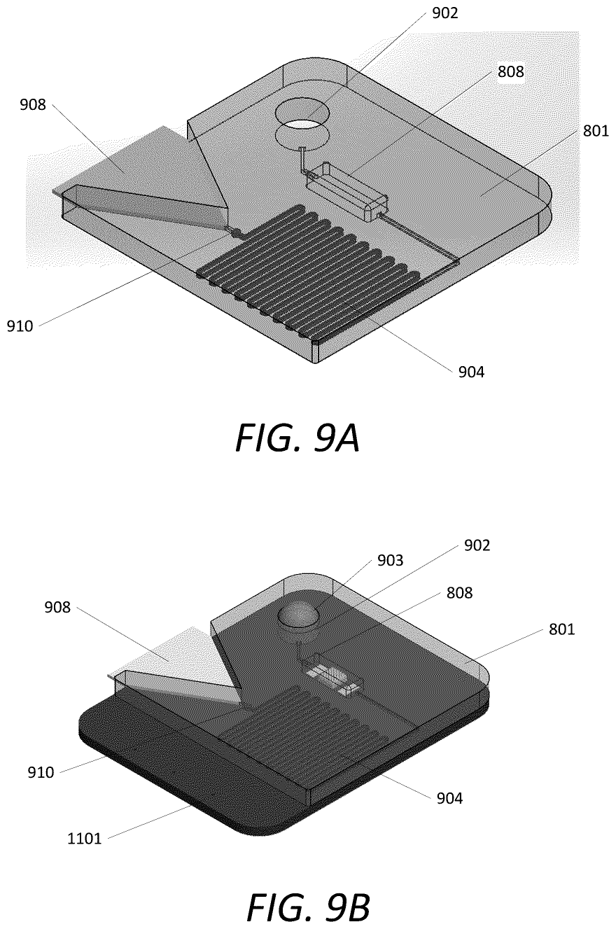

FIGS. 9(A-B) show an exemplary embodiment of the microfluidic element 801 using an absorbent portion to create a vacuum to draw a subject liquid 903 into testing chamber 902. The operational of microfluidic element 801 begins with the introduction of subject liquid 903 at inlet port 902. Upon activation of actuation valve 910, positioned in the open state, a pre-filled sacrificial liquid 904 begins to wick into absorbent material 908. This wicking action induces a negative pressure within testing chamber 808, facilitating the directed flow of the subject liquid 902 from inlet port 902 toward testing chamber 808 where it comes into contact with working electrode 802, counter electrode 804, and reference electrode 806. Microfluidic element 801 can be fabricated using a molding process similar to that used to form the arrays of micropillars. In preferred embodiments, microfluidic element 801 is composed of PDMS.

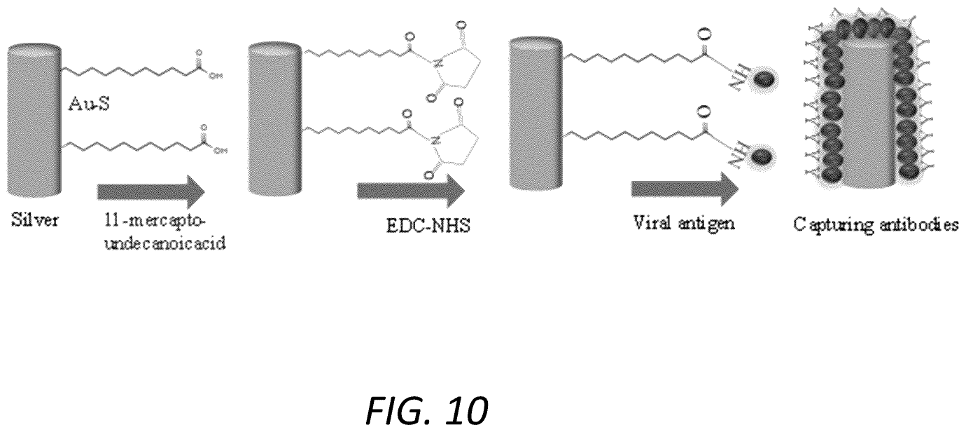

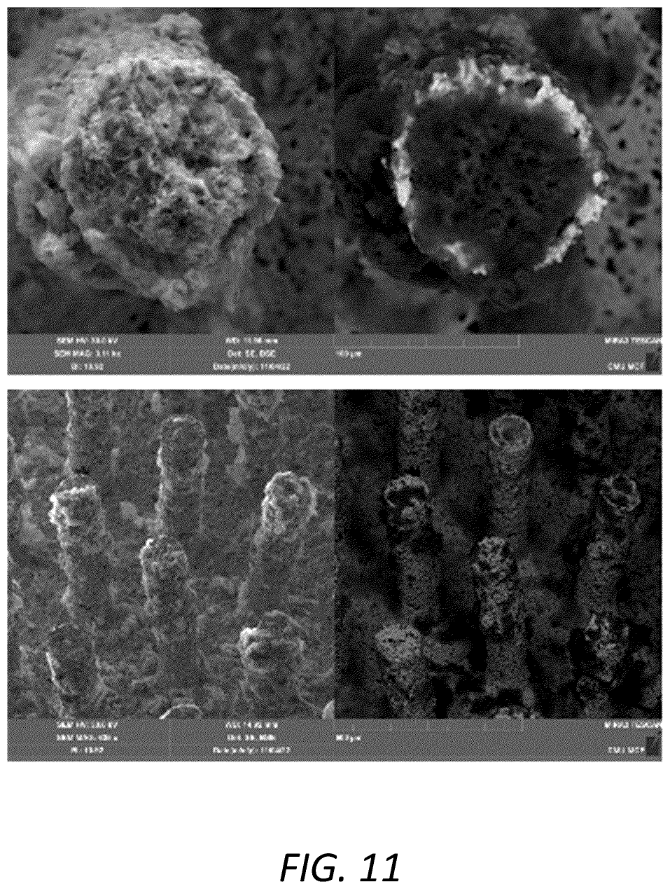

In one embodiment of the invention, the array of micropillars previously described will have the micropillar geometry functionalized by biosensitive nanomaterials such as reduced graphene oxide and specific pathogen proteins by enabling chemistry such as the EDC-NHS chemistry, as shown in FIG. 10. In other embodiments, the specific “sensing molecule” can be replaced with other molecules to detect bioagents, nucleic acids, and other biomarkers (e.g., infection products in the blood, cancer molecules, CSF molecules, PH levels, etc.). A SEM image of the micropillars coated with the nanomaterials is shown in FIG. 11.

As described herein, the POC device and the method of manufacture are advantageous in they allow for the scaled-up manufacture of the device, making the device commercially viable. Furthermore, the embodiment using the PCB makes the device convenient to use, even by a layperson and, as such, may open up markets that may have not been otherwise available.

Although various examples of the method of manufacture and the POC device itself have been presented herein, as would be realized by those of skill in the art, many variations of the manufacturing process and the POC device are possible and are contemplated to be within the scope of the invention.

1. A biosensor comprising:
an electronics module;
a sensing module comprising one or more arrays of micropillars coated with one or more biosensitive materials; and
a microfluidic module for routing one or more subject liquids to the sensing module.

2. The biosensor of claim 1 wherein the electronics module comprises:
a printed circuit board;
one or more reference electrodes defined on the printed circuit board;
one or more counter electrodes defined on the printed circuit board; and
one or more working electrodes defined on the printed circuity board, each working electrode having an array of micropillars coupled thereto.

3. The biosensor of claim 2 wherein the electronics module further comprises one or more of:
circuitry for controlling operation of the biosensor, a power source, one or more indicators and a means of offboarding output of the biosensor.

4. The biosensor of claim 3 wherein the indicators include one or more of LED lights, digital read-outs and a display.

5. The biosensor of claim 3 wherein the means of offboarding comprises one or more of Bluetooth® connectivity and electrical adaptors to external devices.

6. The biosensor of claim 2 wherein the microfluidic module defines one or more microfluidic elements, each microfluidic element defining a testing chamber, each testing chamber containing the working electrode, a reference electrode and a counter electrode.

7. The biosensor of claim 6 wherein each microfluidic element further comprises:
a channel to direct a subject liquid to the testing chamber.

8. The biosensor of claim 7 wherein each microfluidic element has a portion that, when pushed, forces the subject liquid through the channel into the testing chamber and into contact with the working electrode, the reference electrode and the counter electrode.

9. The biosensor of claim 7 wherein the microfluidic element comprises an absorbent material that, when absorbing a sacrificial liquid, creates a vacuum that pulls the subject liquid through the channel into the testing chamber and into contact with the working electrode, the reference electrode and the counter electrode.

10. The biosensor of claim 1 wherein micropillars may vary in size, shape or height within a single array or from array to array.

11. The biosensor of claim 1 wherein micropillars may vary in density, arrangement, porosity or composition from array to array.

12. A method of manufacture of a sensing element of a biosensor comprising:
creating a mastermold comprising a plurality of arrays of micropillars;
creating a negative production mold using the mastermold, the production mold comprising a plurality of cavities defining the shape of the arrays of micropillars;
mixing powdered metal with a binder to yield a slurry;
placing the slurry into each of the plurality of cavities in the production mold;
centrifuging the production mold to force the slurry to fill the cavities to yield a plurality of green arrays;
removing the green arrays from the production mold and
sintering the green arrays.

13. The method of claim 12 wherein the negative production mold is composed of polydimethylsiloxane.

14. The method of claim 12 wherein the binder comprises a mixture of polyvinyl alcohol and a solvent.

15. The method of claim 12 wherein the powered metal comprises particles of one or more conductive, sinterable metals.

16. The method of claim 15 wherein the powered metal comprises one or more of nickel, stainless steel, titanium, copper, copper alloys, gold, gold alloys and silver alloys.

17. The method of claim 12 further comprising:
centrifuging the production mold to facilitate removal of the green arrays from the production mold.

18. The method of claim 12 further comprising:
exposing the production mold to a drying process to facilitate removal of the green arrays from the molds.

19. The method of claim 12 further comprising:
exposing the green devices to a predetermined temperature for a predetermined period of time effective to evaporate the solvent.

20. The method of claim 12 wherein the micropillars in the mastermold are square in cross-sectional shape having sides approximately 150 μm in length and a height of approximately 600 μm.

21. The method of claim 20 wherein the metal power comprises particles of metal in a range of 5 μm in size.

22. The method of claim 12 further comprising:
coating the sintered arrays with one or more biosensitive materials.

23. The method of claim 22 further comprising:
coupling one or more of the coated arrays to one or more working electrodes defined on a printed circuit board.

24. The method of claim 23 further comprising:
defining one or more counter electrodes and one or more reference electrodes on the printed circuit board.

25. The method of claim 24 further comprising:
providing a microfluidic module comprising one or more microfluidic elements to direct a subject liquid to one or more testing chambers, each testing chamber containing a working electrode, a reference electrode and a counter electrode.

26. The method of claim 25 wherein the microfluidic module has a portion that, when pushed, forces the subject liquid into the one or more testing chambers and into contact with the working electrode, the reference electrode and the counter electrode.

27. The method of claim 25 wherein the microfluidic module comprises an absorbent material that, when absorbing a sacrificial liquid, creates a vacuum that pulls the subject liquid into the one or more testing chambers and into contact with the working electrode, the reference electrode and the counter electrode.

28. The method of claim 12 wherein micropillars may vary in size, shape or height within a single array or from array to array.

29. The method of claim 12 wherein micropillars may vary in density, arrangement, porosity or composition from array to array.

30. The method of claim 29 wherein the porosity of the micropillars may be varied by varying a ratio of metal powder to binder in the slurry.

31. The method of claim 29 wherein the porosity of the micropillars may be varied by varying the size of metal particles in the metal powder.

32. A biosensor comprising:
an electronics module;
a sensing module comprising one or more arrays of micropillars manufactured by the method of claim 12; and
a microfluidic module for routing one or more subject liquids to the sensing module.
